(19) United States
(12) Patent Application Publication
(10) Pub. No.: US 2022/0218507 A1
(43) Pub. Date: Jul. 14, 2022

(54) REHABILITATION GLOVE

(71) Applicant: NEOFECT Co., Ltd., Seongnam-si (KR)

(72) Inventors: Sung Gon MOON, Seoul (KR); Byung Geol PARK, Icheon-si (KR); Dong Jun LEE, Seoul (KR); Young Geun CHOI, Yongin-si (KR)

(73) Assignee: NEOFECT Co., Ltd., Seongnam-si (KR)

(21) Appl. No.: 17/712,564

(22) Filed: Apr. 4, 2022

Related U.S. Application Data
(63) Continuation of application No. PCT/KR2020/013546, filed on Oct. 6, 2020.

(30) Foreign Application Priority Data
Oct. 7, 2019 (KR) 10-2019-0124147
Apr. 28, 2020 (KR) 10-2020-0051231

(51) Int. Cl. A61F 5/01

(52) U.S. Cl. CPC A61F 5/0118; A61F 2005/0197

(57) ABSTRACT

Disclosed is a rehabilitation glove including a body surrounding at least a portion of a hand of a user, and a strap unit coupled to the body, and formed to provide a tensile force in a direction, in which a joint of a finger is spread, at a hand back part of the user.

### CROSS-REFERENCE TO RELATED APPLICATIONS

The present application is a continuation of International Patent Application No. PCT/KR2020/013546, filed on Oct. 6, 2020, which is based upon and claims the benefit of priority to Korean Patent Application Nos. 10-2019-0124147 filed on Oct. 7, 2019 and 10-2020-0051231 filed on Apr. 28, 2020. The disclosures of the above-listed applications are hereby incorporated by reference herein in their entirety.

### BACKGROUND

Embodiments of the inventive concept relate to a rehabilitation glove.

A spasticity phenomenon is a phenomenon, in which an entire body of a person is paralyzed mainly due to cerebral strokes or stroke diseases, and refers to a phenomenon, in which a hand, an arm, a leg, and the like of a user is stiffened while being curved. In particular, a user having a spasticity phenomenon in a hand and a finger may be accompanied with pain when he or she closes a hand for a long time, and his or her health may deteriorate due to a nerve problem such as musculoskeletal system disorders.

Accordingly, a user having spasticity in a hand has to perform rehabilitation training for relaxing a finger having spasticity by spreading the finger periodically to reduce pain and prevent diseases from being worsened.

The rehabilitation training is performed by using a rehabilitation glove, and the rehabilitation glove is a glove for users who requires a rehabilitation treatment for alleviating a spasticity phenomenon in a hand and a finger.

The rehabilitation glove may support rehabilitation training of a hand and a finger for minimizing a contracture phenomenon of a finger having spasticity and securing lengths of muscles by extending detachable spasticity alleviating straps formed on upper sides of fingers in a direction, in which the fingers of the user are spread.

However, a rehabilitation glove product utilized conventionally is configured such that joints of fingers are spread at once by using one strap in one finger. Accordingly, it is difficult for a user to sufficiently achieve an effect of training because it is difficult to finely adjust a degree of grasping a hand by himself or herself during a rehabilitation exercise. Furthermore, it may cause an inconvenience in carrying out everyday activities at a time point, at which the spasticity of the user is lowered.

Furthermore, in the conventional rehabilitation glove product, a spasticity alleviating strap is located at an end bone part of a finger, and thus a function of spreading a finger is not minimized so that an end of the hand cannot be properly spread, whereby an efficiency of rehabilitation training for alleviating a spasticity phenomenon of the user is low.

### SUMMARY

Embodiments of the inventive concept provide a rehabilitation glove that may assist motion of a finger.

Embodiments of the inventive concept also provide a rehabilitation glove that may alleviate a spasticity phenomenon of a user by optimizing an operation of spreading a finger by a user who requires a finger rehabilitation exercise and helping properly spreading an end of a hand.

The technical objects of the inventive concept are not limited to the above-mentioned ones, and the other unmentioned technical objects will become apparent to those skilled in the art from the following description.

A rehabilitation glove according to an embodiment of the inventive concept includes a body surrounding at least a portion of a hand of a user, and a strap unit coupled to the body, and formed to provide a tensile force in a direction, in which a joint of a finger is spread, at a hand back part of the user.

Furthermore, the strap unit may include a first strap and a second strap connected to each other side by side along a direction, in which one finger of the user extends, and formed to deliver the tensile force to each other.

Furthermore, one end of the first strap may be coupled to a portion of the body, which surrounds an end of the finger of the user, and an opposite end of the first strap and one end of the second strap may be coupled to be detachably attached to each other.

Furthermore, an opposite end of the second strap may be coupled to be detachably attached to a portion of the body, which covers a hand back or a wrist of the user.

Furthermore, the strap unit may further include a third strap disposed side by side with the first strap and the second strap, an opposite end of the second strap and one end of the third strap may be coupled to be detachably attached to each other, and an opposite end of the third strap may be coupled to be detachably attached to a portion of the body, which covers the hand back or the wrist of the user.

Furthermore, the opposite end of the first strap and the one end of the second strap may be attached to overlap each other.

Furthermore, the second strap may include a support ring supported by the body to surround the finger of the user at one end thereof, which is coupled to the first strap.

Furthermore, the support ring may include a first ring part formed to surround a portion of a circumference of the finger of the user, and a second ring part formed to surround the remaining portions of the circumference of the finger of the user, and coupled to the first ring part to cross the first ring part at a preset angle.

Furthermore, the first strap and the second strap may be formed of a material having an elastic coefficient of 167 N/m² to 289 N/m².

Furthermore, the strap unit may include a finger strap formed to deliver the tensile force along a direction, in which one finger of the user extends, and a hand back strap, in which an end of the finger strap is coupled to one side surface thereof and an opposite side surface of which is coupled to a surface of the body to deliver the tensile force of the finger strap to the body.

Furthermore, the body may include a thimble part formed to surround an end of the finger of the user and coupled to one end of the finger strap.

Furthermore, the body may include guide parts disposed to be spaced apart from each other in a direction, in which the finger of the user extends, and coupled to a surface of the body such that the finger strap is interposed therebetween.

Furthermore, the finger strap may be formed of a material having an elastic coefficient of 167 N/m² to 289 N/m².

Furthermore, the strap unit may include a finger spreading part connected to the body and that spreads and fixes a finger of the user, and a finger fixing part that pulls and fixes the finger that is spread by the finger spreading part, and the finger spreading part may include a support member that spreads and fixes a bone part of the finger of the user.

Furthermore, the support member may support the finger while contacting a back of the finger or a bottom of the finger of the user.

Furthermore, the finger spreading part may spread the knuckle of the finger by applying a force in a direction from a palm toward a hand back of the user.

Furthermore, the finger fixing part may be connected to the body, and the finger fixing part may include a Velcro fastener, and is attached to the body to fix the spread finger.

Furthermore, the finger fixing part may have elasticity.

Furthermore, the finger spreading part may include a Velcro fastener, and is attached to the finger fixing part to spread the finger of the user.

Furthermore, the finger fixing part may be connected to the body, and the finger fixing part may include a Velcro fastener, and is attached to the body to fix the spread finger.

Furthermore, the finger fixing part may be disposed to extend in a direction from an end of the finger toward a hand back of the user.

Furthermore, the rehabilitation glove may further include a glove part surrounding the body, and the glove part may include a hand back part surrounding a hand back of the user and a back of the finger, and a palm part surrounding a bottom of the finger of the user and exposing at least a portion of a palm.

Furthermore, a hand back part of the body may include a Velcro fastener, and is detachably attached to the finger fixing part.

Furthermore, the glove part may further include a wrist fixing part surrounding a wrist.

Furthermore, the wrist fixing part may include a Velcro fastener.

The above-described solutions are merely exemplary, and should not be construed as an intention to limit the inventive concept. In addition to the above-described exemplary embodiments, additional embodiments described in the drawings and the detailed description of the inventive concept may be present.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating a rehabilitation glove according to a first embodiment of the inventive concept;

FIG. 2 is a side view of the rehabilitation glove according to the first embodiment of the inventive concept;

FIG. 3 is a side view of a strap unit of the rehabilitation glove according to the first embodiment of the inventive concept;

FIG. 4 is a view of a support ring of the strap unit of the rehabilitation glove according to the first embodiment of the inventive concept;

FIG. 5 is a view illustrating a rehabilitation glove according to a second embodiment of the inventive concept;

FIGS. 6A, 6B, and 6C are views illustrating an embodiment of a hand back strap of a strap unit of the rehabilitation glove according to the second embodiment of the inventive concept;

FIG. 7 is a graph depicting an experimental result of elasticity of the strap units in the first embodiment and the second embodiment of the inventive concept;

FIG. 8A is a schematic view of a rehabilitation glove according to a third embodiment of the inventive concept, which is put on, when the rehabilitation glove is viewed from a hand back;

FIG. 8B is a schematic view of the rehabilitation glove according to the third embodiment of the inventive concept, which is put on, when the rehabilitation glove is viewed from a palm;

FIG. 9A is a schematic view of a glove part of the rehabilitation glove according to the third embodiment of the inventive concept, which is put on, when the glove part is viewed from a hand back;

FIG. 9B is a schematic view of the glove part of the rehabilitation glove according to the third embodiment of the inventive concept, which is put on, when the glove part is viewed from a palm;

FIG. 10A is a schematic view of a hand insertion part of the rehabilitation glove according to the third embodiment of the inventive concept, which is put on, when the hand insertion part is viewed from a hand back;

FIG. 10B is a view schematically illustrating a cross-section of area A of FIG. 10A;

FIG. 11A is a schematic view of a hand insertion part included in a rehabilitation glove according to a fourth embodiment of the inventive concept, when the hand insertion part is viewed from a hand back while a finger spreading part is not fixed;

FIG. 11B is a schematic view of the hand insertion part included in the rehabilitation glove according to the fourth embodiment of the inventive concept, when the hand insertion part is viewed from the hand back while the finger spreading part is fixed; and

FIG. 11C is a view schematically illustrating a cross-section of area A of FIG. 10B.

### DETAILED DESCRIPTION

Hereinafter, exemplary embodiments of the inventive concept will be described in detail with reference to the accompanying drawings so that those skilled in the art to which the inventive concept pertains may easily carry out the inventive concept. However, the inventive concept may be implemented in various different forms, and is not limited to the embodiments. Further, to clearly describe the inventive concept, parts that are irrelevant to the description are excluded from the drawings and the similar parts are denoted by similar reference numerals throughout the specification.

Throughout the specification, when it is described that a part is “connected” to another part, it means not only that they are ‘directly connected’ to each other but also that they ‘are electrically connected to each other while another element is interposed therebetween’. Furthermore, it should be understood that when a part includes an element, it means that another element is not excluded but may be further included unless particularly described in an opposite way, and presence or additional possibility of one or more other features, numbers, steps, operations, elements, and partial products, or combinations thereof is not excluded in advance.

Hereinafter, various embodiments of the inventive concept will be described with reference to the accompanying drawings.

A rehabilitation glove according to various embodiments of the inventive concept is directed to alleviating pain of a user having spasticity in hands and fingers and preventing a phenomenon, in which muscles and nerve diseases deteriorate, and is configured to minimize a contracture phenomenon of hands and fingers of a user having spasticity and allow rehabilitation training of an entire palm of a hand or an entire bottom of a finger.

### First Embodiment

Hereinafter, a rehabilitation glove according to a first embodiment of the inventive concept will be described.

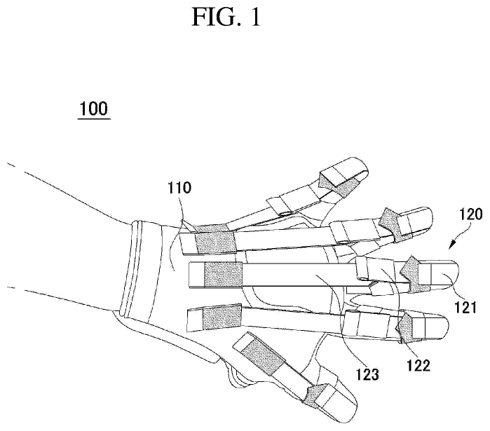

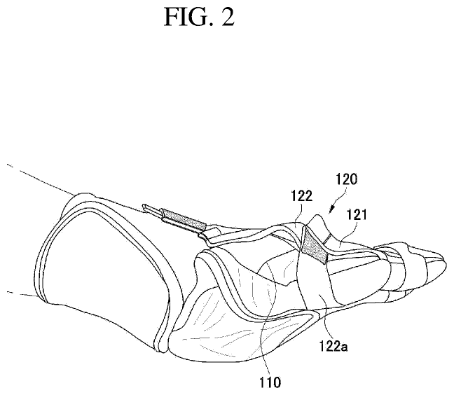

FIG. 1 is a view illustrating the rehabilitation glove according to the first embodiment of the inventive concept. FIG. 2 is a side view of the rehabilitation glove according to the first embodiment of the inventive concept.

Referring to FIGS. 1 and 2, a rehabilitation glove 100 according to the first embodiment of the inventive concept includes a body 110 formed to cover a hand of a user (a wearer) such that the user puts on the rehabilitation glove 100, and a strap unit 120 coupled to the body 110, and formed to provide a tensile force in a direction, in which a knuckle of the user is spread at a hand back portion of the user.

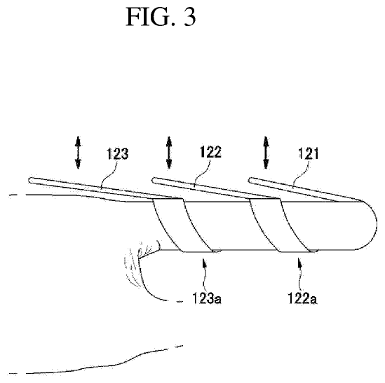

The strap unit 120 formed on an upper side of the finger or the hand back portion located in the body 110, as illustrated in FIGS. 1 and 2, includes a first strap 121 and a second strap 122 connected side by side along a direction, in which one finger of the user extends to deliver a tensile force for spreading a knuckle. Furthermore, the strap unit 120 may further include a third strap 123 disposed side by side with the first strap 121 and the second strap 122. Here, the extension direction of the finger may be any one of opposite directions, in which a portion of the body that accommodates the finger of the user extend, and in the present embodiment, may refer to a direction (a direction that faces a left side from a right side of FIG. 3, which will be described below) that faces the hand back from an end of the finger.

In more detail, in the rehabilitation glove 100, a plurality of strap units 120 may be formed in one finger, that is, the first strap 121, the second strap 122, and the third strap 123 may be disposed at finger bone parts and a hand back portion.

According to the embodiment of the inventive concept, the first strap 121 may be disposed in a distal bone part of the finger, the second strap 122 may be disposed in a bone part that is next to the bone part, in which the first strap 121 is disposed, and the third strap 123 may be disposed over a hand back portion in a bone part that is next to the bone part, in which the second strap 122 is disposed. Accordingly, because the user may pull the first strap 121, the second strap 122, and the third strap 123 connected according to the bone part portions of the finger by applying a force to them, the user may adjust strengths applied to the bone parts differently according to spasticity levels. In addition, the user may pull and spread the finger having spasticity by applying forces that are lower than when one entire strap is pulled at once, to the first to third straps 121 to 123 one by one.

FIG. 2 is an illustration of an embodiment, in which the strap unit 120 is disposed at a thumb portion of the user, and as in FIGS. 1 and 2, the strap unit 120 may be designed such that two or three straps are disposed in one finger according to a length of a finger and lengths of the straps.

FIG. 3 is a side view of a strap unit of the rehabilitation glove according to the first embodiment of the inventive concept.

In a more detailed description of the first strap 121 according to the embodiment of the inventive concept through FIG. 3, one end of the first strap 121 is coupled to a portion of the body 110, which surrounds an end of the finger of the user, and an opposite end of the first strap 121 is disposed at a location, at which it contacts one end, at which the second strap 122 starts, to be coupled to be detachably attached to the second strap 122. An opposite end of the second strap 122 may be coupled to be detachably attached to a portion of one end of the third strap 123 or the body 110, which covers a hand back or a wrist of the user.

According to the embodiment of the inventive concept, the opposite end of the first strap 121 and the one end of the second strap 122 may be attached to each other to overlap each other.

In more detail, the one end of the first strap 121 may be located at an end of the finger in the body 110 to be fixedly connected to the body 110, and a Velcro fastener may be formed at the opposite end of the first strap 121 to be detachably attached to an upper surface of the second strap 122. Similarly, the one end of the second strap 122 also may be fixedly connected to the body 110, and a Velcro fastener may be formed at the opposite end of the second strap 122 such that the opposite end of the second strap 122 may be detachably attached to the third strap 123 or the body 110. When the third strap 123 is further included, the one end of the third strap 123 may be coupled to the body 110, and the opposite end of the third strap 123, at which a Velcro fastener is formed, may be detachably attached to the body 110. According to the configuration, the strap unit 120 may be configured such that the straps are connected to each other side by side without any broken portion to deliver a force such that all the parts ranging from the finger bone parts to the palm are relaxed. Meanwhile, a portion of a surface or an entire surface of the body 110 may be formed of a material, to which the Velcro fasteners of the first to third straps 121, 122, and 123 may be detachably attached.

As described above, because ends of the first strap 121 and the second strap 122 are disposed side by side to overlap each other, the user may sequentially detach the opposite end of the first strap 121 from the body 110 or the second strap 122, extend the first strap 121 such that the first bone part of the finger is spread, attach the first strap 121 to the second strap 122, detach the second strap 122 from the body 110 or the third strap 123, extend the second strap 122 such that the next bone part of the finger is spread, and attach the second strap 122 to the body 110. Similarly, the third strap 123 may be detached from the body 110, and the third strap 123 may extend such that the entire finger and the entire palm are spread and may be attached to the body 110 again.

In this way, because the first strap 121, the second strap 122, and the third strap 123 are sequentially detached and are attached again such that the bone parts of the finger, the finger, and the wrist are spread, magnitudes of tensile forces of the first strap 121, the second strap 122, and the third strap 123 disposed at the corresponding locations may be adjusted according to spasticity levels and degrees for the finger bone parts. Accordingly, the rehabilitation glove 100 according to the inventive concept may optimize a function of spreading the finger of the user having spasticity as a whole, according to situations.

In detail, the user may carry out rehabilitation training of the bone parts of a finger, as well as the finger of the desired portion, through the strap unit 120 including the plurality of straps. For example, the portions of the bone parts of the finger having spasticity may be relaxed and alleviated by pulling the first strap 121 and the second strap 122, and the portions of the fingers and the palm having the spasticity may be relaxed and alleviated by utilizing the third strap 123.

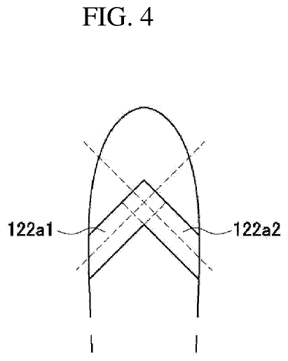

Meanwhile, the one end of the first strap 121 is directly connected to the body 110, but the ends of the second strap 122 and the third strap 123, as illustrated in FIG. 3, may be connected to the body 110 through support rings 122a and 123a. The support rings 122a and 123a have ring shapes that surround the finger, such that the ends of the second strap 122 and the third strap 123 are attached to outsides thereof and inner sides thereof are attached to the body 110. Detailed shapes and functions of the support rings 122a and 123a will be described further with reference to FIG. 4.

FIG. 4 is a view of the support ring of the strap unit of the rehabilitation glove according to the first embodiment of the inventive concept.

Referring to FIGS. 3 and 4, the second strap 122 may include the support ring 122a supported by the body 110 such that the finger of the user is surrounded at the one end thereof coupled to the first strap 121. The third strap 123 also may include the support ring 123a supported by the body 110 such that the finger of the user is surrounded at the one end thereof coupled to the second strap 122.

The support ring 122a according to the embodiment of the inventive concept, as illustrated in FIG. 1, may have a first ring part 122a1 formed to surround a portion of a circumference of the finger of the user, and a second ring part 122a2 formed to surround the remaining portions of the circumference of the finger of the user and coupled to the first ring part 122a1 to cross the first ring part 122a1 at a preset angle.

The support ring 123a coupled to the one end of the third strap 123 also may include a first ring part (not illustrated) formed to surround a portion of a circumference of the finger of the user, and a second ring part (not illustrated) coupled to the first ring part (not illustrated) to cross the first ring part (not illustrated) at a preset angle.

With the configuration, the support rings 122a and 123a, as illustrated in FIG. 3, may be disposed in an inclined form, in which they are inclined with respect to the extension direction of the finger when viewed from a side. With the configuration, even when the user pulls the second strap 122 and the third strap 123 along a surface of the body 110 (along a horizontal direction of FIG. 3) to relax the hand and the finger, the first ring part 122a1 and the second ring part 122a2 may effectively endure a tensile force while a state, in which they are adhered to the body 110, is maintained.

### Second Embodiment

Hereinafter, a rehabilitation glove according to a second embodiment of the inventive concept will be described.

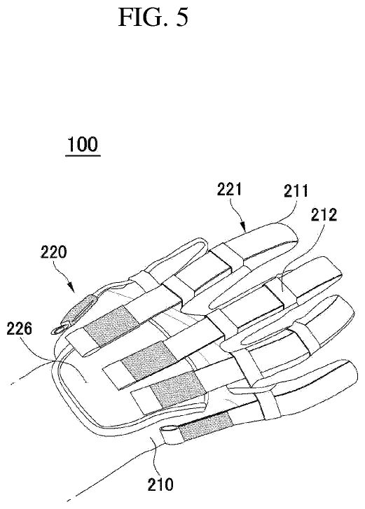

FIG. 5 is a view illustrating an entire appearance of the rehabilitation glove according to the second embodiment of the inventive concept.

A rehabilitation glove 200 according to the second embodiment of the inventive concept includes a body 210 formed to cover a hand of the user to be mounted on the hand, and a strap unit 220 coupled to the body 210 and formed to provide a tensile force in a direction, in which a joint of the finger of the user is spread at the hand back portion of the user.

In detail, the strap unit 220 may include a finger strap 221 formed to deliver a tensile force along a direction, in which one finger of the user extends, and a hand back strap 226, in which an end of the finger strap 221 is coupled to one side surface thereof and an opposite surface of which is coupled to a surface of the body 210 such that a tensile force of the finger strap 221 is delivered to the body 210.

Furthermore, the body 210 according to another embodiment of the inventive concept may include a thimble part 211 formed to surround an end of a finger of the user and coupled to one end of the finger strap 221. One end of the finger strap 221 may be fixedly connected to the body 110, and an opposite end thereof may include a Velcro fastener to be detachably attached to the body 110 or the hand back strap 226 by the thimble part 211. In more detail, an opposite end of the finger strap 221 may be detachably attached to the hand back strap 226 to overlap the hand back strap 226. Accordingly, the finger strap 221 and the hand back strap 226 may be connected to each other to relax the fingers and the palm as a whole.

The one end of the finger strap 221, as illustrated in FIG. 5, is located at a portion of the body 110 corresponding to an end of the hand to perform rehabilitation training of completely spreading the hand of the user. Accordingly, the user may completely relax and spread all portions of the fingers and the palm having spasticity by utilizing the rehabilitation glove 100.

Moreover, the user may relax and alleviate portions of the fingers having spasticity by pulling the finger strap 221, and may relax and alleviate portions of all the fingers and the palm having spasticity by utilizing the hand back strap 226.

Furthermore, according to another embodiment of the inventive concept, the body 210 may further include a guide part 212 formed at a portion of a finger. The plurality of guide parts 212 may be disposed to be spaced apart from each other along the extension direction of the finger of the user, and the finger strap 221 may be interposed between the guide part 212 and the body 210.

In more detail, the guide part 212 may be disposed at the bone part of the finger, and may uniformly deliver a force of spreading the entire finger as the force is delivered to the bone part of the finger by the guide part 212 when the finger strap 221 is pulled. For example, as illustrated in FIG. 5, two guide parts 212 may be disposed in each bone part of the finger, and may be disposed between the first and second bone parts and may be disposed between the second and third bone parts. Furthermore, the plurality of guide parts 212 may fix and support the finger strap 221 such that the finger strap 221 does not deviates to a side of the finger when the user closes the hand.

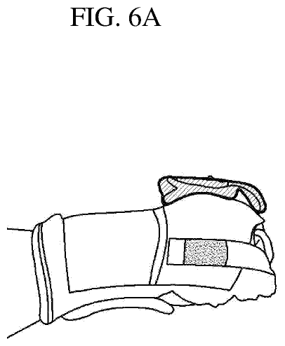

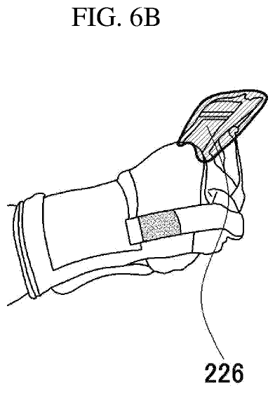

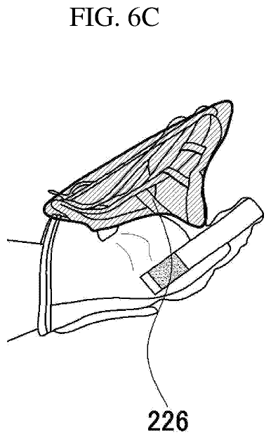

FIGS. 6A, 6B, and 6C are views illustrating an embodiment of a hand back strap of a strap unit of the rehabilitation glove according to the second embodiment of the inventive concept.

According to the hand back strap 226, rehabilitation training of spreading the finger and the hand back at once by pulling the plurality of finger straps 221 at once may be performed by detachably attaching the hand back strap 226 to the body 110.

FIG. 6A illustrates a state, in which the finger straps 221 are attached to the hand back strap 226 while the user closes the hand. The user may detach the hand back strap 226 from the body as illustrated in FIG. 6B, and may pull the hand back strap 226 to the hand back portion of the body toward the body of the user and attach the hand back strap 226 as illustrated in FIG. 6C to carry out rehabilitation training of relaxing the all portions of the fingers and the palm at once or relaxing the wrist toward the hand back.

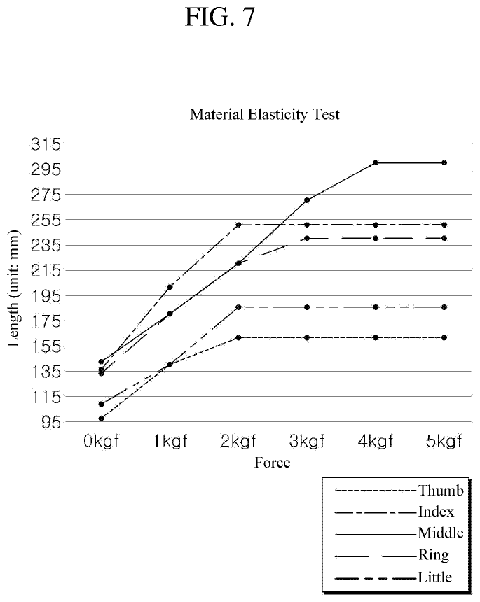

FIG. 7 is a graph depicting an experimental result of elasticity of the strap units in the first embodiment and the second embodiment of the inventive concept.

Hereinafter, designs of the materials of the first to third straps 121 to 123 and the finger strap 221 according to the inventive concept will be described.

First, ranges of forces that are necessary for spreading fingers for rehabilitation exercises may be classified according to symptom levels of users, and may be generally classified into six stages according to Brunnstrom recovery stages (BRSs).

In more detail, stage 1 is a flaccid paralysis stage, in which voluntary movement of all legs is impossible, and stage 2 is a stage, in which associated movement or comitance of a basic leg appears as spasticity is gradually developed so that slight autokinesis is possible. Stage 3 is a stage, in which spasticity is strongest, and the user may voluntarily perform comitance. In stage 4, several voluntary movements separated from comitance may be possible as spasticity of the user gradually decreases, in stage 5, more difficult movements may be performed as comitance of arms and legs of the user is lost, and in stage 6, spasticity of the user disappears and individual joint movements are restored to appear normal.

Meanwhile, a user corresponding to stage 1 may require a force of 0 kgf to 1.5 kgf to spread a finger, a user corresponding to stages 2 and 4 may require a force of 0.5 kgf to 3 kgf, and a user corresponding to stage 3 may require a force of 2 kgf to 3.5 kgf. Stages 5 and 6 are stages, in which stiffening of the user disappears, and thus a force of a specific range may not be required.

Based on this, a material, by which a tensile force may be shown at a length of the same level as those of the fingers, may be applied. Because a range of maximum values, by which grasping power may be show, is 0 kgf to 3.5 kgf, to apply a material having an elastic coefficient that is suitable for a spasticity degree of most of nerve damaged users, referring to FIG. 7, a range of elastic coefficients of the first strap 121 and the second strap 122 may be set to 167 N/m² to 289 N/m² (0.76 MPa to 0.86 MPa).

A result of FIG. 7 is a measurement result obtained by pulling the straps 121, 122, 123, and 221 of the rehabilitation gloves 100 and 200 according to the inventive concept to a maximally prolonged length by using a tensile tester (for example, a push-pull gauge). Forces N (kgf) at maximum lengths may be identified on a transverse axis, and prolonged lengths (mm) of the straps may be identified on a longitudinal axis.

A material elasticity test was performed while forces N (kgs) are increased until the straps were deformed, in which lengths of the straps for the fingers are 97 mm for a thumb, 136 mm for an index finger, 142 mm for a middle finger, 132 mm for a ring finger, and 109 mm for a little finger, before the test, and lengths of the straps for the fingers are 103 mm for a thumb, 140 mm for an index finger, 149 mm for a middle finger, 139 mm for a ring finger, and 113 mm for a little finger, after the test. As a whole, the lengths are increased by 4 mm to 7 mm, and are restored by 3 mm to 4 mm after the straps are left as they are for about two days after the test.

As may be identified in a graph of FIG. 7, a maximum length may be maintained when a pulling force is 2 kgf to 4 kgf, and also a prolonged length is the same as a maximally prolonged length even when the pulling force is up to 15 kgf.

Accordingly, finger rehabilitation training may be performed by setting elastic coefficients of the straps 121, 122, 123, and 221 to 167 N/m² to 289 N/m² and repeating an act of spreading the finger by pulling the first to third straps 121 to 123, and the finger strap 221 and bending and spreading the finger by the user.

Furthermore, the strap units 120 and 220 may be formed of a flexible material, and may be formed of materials such as silicon, span, neoprene, and rubber. Rehabilitation training intensity may be secured by an elastic force provided by the flexible straps.

According to the rehabilitation gloves 100 and 200, the strap units 120 and 220 include the first strap 121, the second strap 122, the third strap 123, the finger strap 221, and the hand back strap 226, whereby rehabilitation training is helped to be performed with no difficulty as the user wears the rehabilitation glove 100 in everyday lives. Moreover, general exercises and rehabilitation training may be performed together as the wearer wears the rehabilitation glove 100 in cycling, outdoor activities, and other exercises using sport outfits.

In particular, the rehabilitation glove 100 according to the inventive concept, unlike an existing product, may include the strap unit 220 including the first strap 121, the second strap 122, and the third strap 123 that are classified according to the bone parts of the finger, and may perform rehabilitation training of spreading and relaxing the hand and the fingers having spasticity one by one and completely spreading the entire palm, by the user, while different intensities are applied to the classified areas.

### Third Embodiment

Hereinafter, a rehabilitation glove according to a third embodiment of the inventive concept will be described.

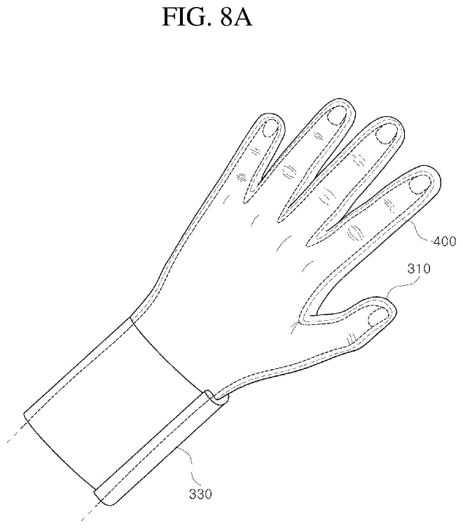

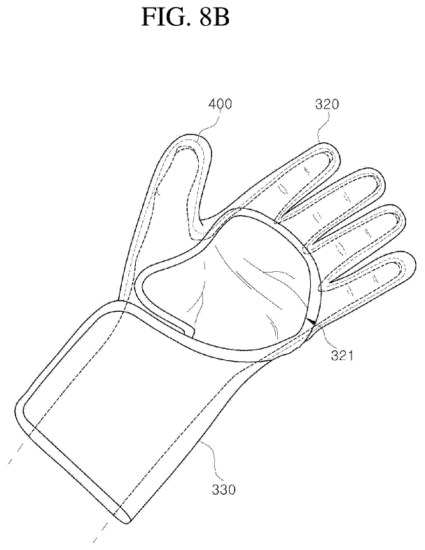

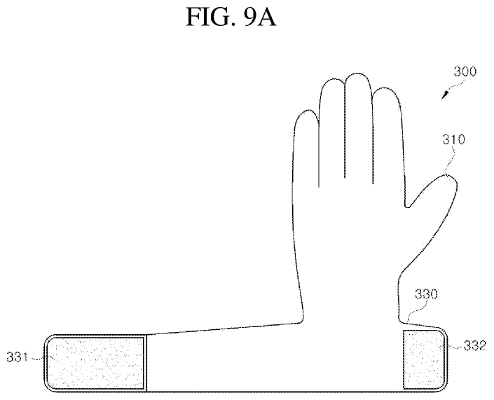

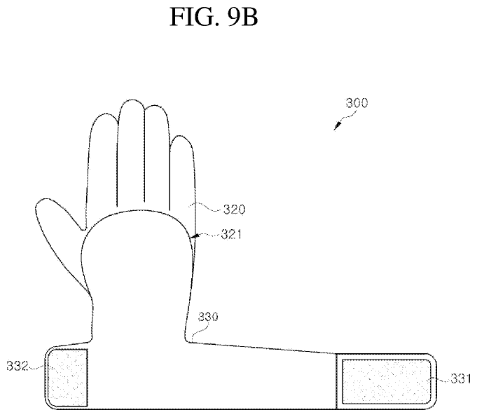

FIG. 8A is a schematic view of the rehabilitation glove according to the third embodiment of the inventive concept, which is put on, when the rehabilitation glove is viewed from a hand back. FIG. 8B is a schematic view of the rehabilitation glove according to the third embodiment of the inventive concept, which is put on, when the rehabilitation glove is viewed from a palm. FIG. 9A is a schematic view of a glove part of the rehabilitation glove according to the third embodiment of the inventive concept, which is put on, when the glove part is viewed from a hand back. FIG. 9B is a schematic view of the glove part of the rehabilitation glove according to the third embodiment of the inventive concept, which is put on, when the glove part is viewed from a palm.

Referring to FIGS. 8A, 8B, 9A, and 9B, the rehabilitation glove according to the third embodiment of the inventive concept includes a glove part 300 and a hand insertion part 400.

The glove part 300 surrounds the hand insertion part 400. The glove part 300 exposes at least a portion of a palm of the user. The glove part 300 may expose at least a portion of the palm of the user to allow the user to feel a haptic feeling of an object with the palm part.

The glove part 300 includes a hand back part 310, a palm part 320, and a wrist fixing part 330. The hand back part 310, the palm part 320, and the wrist fixing part 330 may be integrally formed.

The hand back part 310 may surround the hand back and backs of the fingers of the user. The palm part 320 may surround bottoms of the fingers and expose at least a portion of the palm. The palm part 320 may include a palm exposure hole 321. At least a portion of the palm of the user may be exposed through the palm exposure hole 321. The palm part 320 may expose at least a portion of the palm of the user to allow the user to feel a haptic feeling of an object with the palm part.

The wrist fixing part 330 may surround a wrist of the user. A size of the wrist fixing part 330 may be adjusted according to the wrist of the user.

The wrist fixing part 330 may be formed of a porous material.

The wrist fixing part 330 may spread and fix a bent wrist. For example, the wrist fixing part 330 may wind and fix a bent portion of the wrist.

The wrist fixing part 330 may include Velcro fasteners 331 and 332. For example, the Velcro fasteners 331 and 332 may be disposed on an inner skin of the wrist fixing part 330. A Velcro fastener attaching/detaching part, to which a finger fixing part 430 is detachably attached and which will be described below, may be disposed on an outer skin of the wrist fixing part 330.

The size of the wrist fixing part 330 may be adjusted according to the wrist of the user, and may be detachably attached band fixed by the Velcro fasteners 331 and 332. Although FIGS. 9A and 9B illustrate as an example that the wrist fixing part 330 includes the two Velcro fasteners 331 and 332, the inventive concept is not limited thereto, and the entire wrist fixing part 330 may include one Velcro fastener. Furthermore, the wrist fixing part 330 may include three or more Velcro fasteners.

The user may insert a hand into the hand insertion part 400. The user may insert the hand into the hand insertion part 400 to put on the rehabilitation glove. The glove part 300 may surround the hand insertion part 400.

The hand insertion part 400 surrounds the hand back of the user. The hand insertion part 400 may expose at least a portion of the palm of the user. The hand insertion part 400 surrounds the finger of the user. The hand insertion part 400 will be described later in more detail.

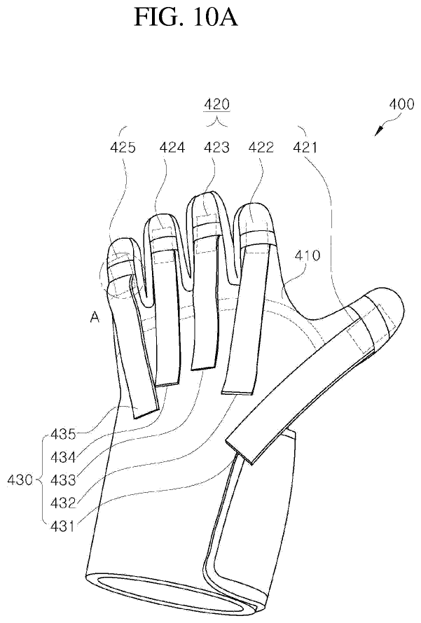

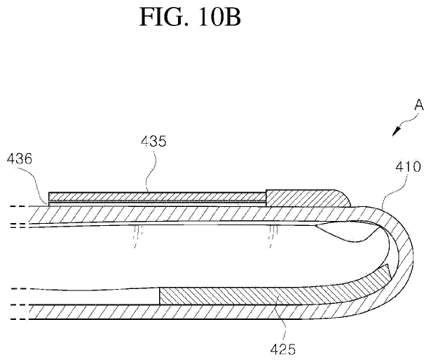

FIG. 10A is a schematic view of the hand insertion part included in the rehabilitation glove according to the third embodiment of the inventive concept, when the hand insertion part is viewed from the hand back. FIG. 10B is a view schematically illustrating a cross-section of area ‘A’ of FIG. 10A.

Referring to FIGS. 8A to 10B, the hand insertion part 400 includes a body 410 and a strap unit.

The body 410 surrounds at least a portion of the hand of the user. The body 410 surrounds the hand back of the user. The body 410 may expose at least a portion of the palm of the user. The body 410 may surround the finger of the user. The body 410 may expose at least a portion of the palm of the user to allow the user to feel a haptic feeling of an object with the palm part.

The body 410 may be formed of a porous material.

The strap unit is coupled to the body 410 and is formed to provide a tensile force in a direction, in which the joints of the finger of the user are spread at the hand back pat of the user, and includes a finger spreading part 420 and the finger fixing part 430.

The finger spreading part 420 spreads and fixes the finger of the user. The finger spreading part 420 is provided on an outer surface or an inner surface of the body 410. The finger spreading part 420, for example, may surround the finger and spread and fix the finger, or may spread the finger by applying a force to the finger in a direction from a bottom part to a back of the finger and fix the finger.

The finger spreading part 420 includes a thumb spreading part 421, an index finger spreading part 422, a middle finger spreading part 423, a ring finger spreading part 424, and a little finger spreading part 425. The thumb spreading part 421 spreads and fixes a thumb of the user. The index finger spreading part 422 spreads and fixes an index finger of the user. The middle finger spreading part 423 spreads and fixes a middle finger of the user. The ring finger spreading part 424 spreads and fixes a ring finger of the user. The little finger spreading part 425 spreads and fixes a little finger of the user. An embodiment of the finger spreading part 420 will be described later in more detail.

The finger fixing part 430 pulls and fixes the finger spread by the finger spreading part 420. The finger fixing part 430 is connected to the body 410. The finger fixing part 430 may be disposed to extend in a direction from an end of the finger toward the hand back.

The finger fixing part 430 includes a thumb fixing part 431, an index finger fixing part 432, a middle finger fixing part 433, a ring finger fixing part 434, and a little finger fixing part 435. The thumb fixing part 431 pulls and fixes the spread thumb of the user. The index fixing part 432 pulls and fixes the spread index finger of the user. The middle fixing part 433 pulls and fixes the spread middle finger of the user. The ring fixing part 434 pulls and fixes the spread ring finger of the user. The little fixing part 435 pulls and fixes the spread little finger of the user. An embodiment of the finger fixing part 430 will be described later in more detail.

As an embodiment, referring to FIGS. 10A and 10B, the finger spreading parts include support members 421, 422, 423, 424, and 425 that spread and fix knuckles of the fingers of the user.

Any support member 421, 422, 423, 424, and 425 is possible without specific limitation as long as it may spread and fix the knuckles of the fingers, but for example, may include a steel plate, plywood, plastic, and the like.

The support members 421, 422, 423, 424, and 425 may include an elastic material. For example, the support members 421, 422, 423, 424, and 425 may include an elastic material, and may provide an effect of spreading and fixing the bone parts of the fingers and alleviating the spasticity of the user more softly.

The support members 421, 422, 423, 424, and 425, for example, may spread and fix the bone part that is closest to an end of the finger of the user. The support members 421, 422, 423, 424, and 425, for example, may spread and fix the bone part next to the bone part that is closest to the end of the finger of the user. The support members 421, 422, 423, 424, and 425, for example, may spread and fix both of the bone part that is closest to the end of the finger and the bone part next to the bone part that is closest to the end of the finger.

The support members 421, 422, 423, 424, and 425 may be disposed at finger portions of the body 410. The support members 421, 422, 423, 424, and 425 may be fixed to the finger portions of the body 410. The support members 421, 422, 423, 424, and 425 may be disposed at finger portions of the body 410 corresponding to the knuckles of the finger of the user.

Although FIGS. 10A and 10B illustrate as an example that the support members 421, 422, 423, 424, and 425 contact the bottoms of the fingers, the inventive concept is not limited thereto, and the support members 421, 422, 423, 424, and 425 may contact the backs of the fingers.

The support members 421, 422, 423, 424, and 425 may include the first support member 421, the second support member 422, the third support member 423, the fourth support member 424, and the fifth support member 425. The first support member 421 may spread and fix the knuckles of the thumb of the user. The second support member 422 may spread and fix the knuckles of the index finger of the user. The third support member 423 may spread and fix the knuckles of the middle finger of the user. The fourth support member 424 may spread and fix the knuckles of the ring finger of the user. The fifth support member 425 may spread and fix the knuckles of the little finger of the user.

The finger spreading part may include support members 421, 422, 423, 424, and 425, and for example, may spread the knuckles of the fingers by applying a force in a direction from the palm toward the hand back of the user.

However, the inventive concept is not limited thereto, and the finger spreading part may include the support members 421, 422, 423, 424, and 425, and for example, the knuckles of the fingers may be spread by applying a force in a direction from the hand back toward the palm of the user. Then, the rehabilitation glove according to the third embodiment of the inventive concept may further include a separate support member that supports a finger in a direction of a bottom of the finger.

The finger fixing part 430 includes a Velcro fastener 436. The finger fixing part 430 is connected to the body 410. The finger fixing part 430 may include the Velcro fastener 436, and may be attached to the body 410 to fix the spread fingers with the support members 421, 422, 423, 424, and 425.

The Velcro fastener 436 may include a first Velcro fastener, a second Velcro fastener, a third Velcro fastener, a fourth Velcro fastener, and a fifth Velcro fastener. The first Velcro fastener may be disposed in the thumb fixing part 431. The second Velcro fastener may be disposed in the index finger fixing part 432. The third Velcro fastener may be disposed in the middle finger fixing part 433. The fourth Velcro fastener may be disposed in the ring finger fixing part 434. The fifth Velcro fastener may be disposed in the little finger fixing part 435.

The finger fixing part 430 is detachably attached to the body 410. The finger fixing part 430 may be attached to the body 410 by applying a force in a direction from an end of the finger toward the wrist. The finger fixing part 430, for example, may be detachably attached to the hand back part of the body 410. The hand back part of the body 410 may include a Velcro fastener.

The finger fixing part 430 may have elasticity. The finger fixing part 430 may have elasticity, and thus may allow the user having spasticity to spread the hand and perform a finger control training (for example, training of grasping the fingers).

In a description of the embodiment illustrated in FIGS. 10A and 10B as an example, the fingers of the user may be spread by applying a force in a direction from the palm toward the hand back by the finger spreading part 420 including the support members. The finger spread by the finger spreading part 420 may be fixed by pulling the finger fixing part 430 connected to the body 410 in a direction from an end of the finger toward the wrist and attaching the finger fixing part 430 to the body 410. Accordingly, the rehabilitation glove according to the third embodiment of the inventive concept may spread and fix the finger of the user.

The rehabilitation glove according to the third embodiment of the inventive concept may include the glove part 300 having a shape of a mitten. In more detail, the glove part 300 may include a first glove part, into which a thumb is inserted, and a second glove part, into which the remaining fingers (an index finger, a middle finger, a ring finger, and a little finger) are inserted.

The finger spreading part 420 may include a thumb finger spreading part and a remaining finger spreading part. The thumb of the user may be spread by applying a force in a direction from the palm toward the hand back with the thumb spreading part including the thumb support member that supports the thumb. The remaining fingers of the user may be spread by applying a force in a direction from the palm toward the hand back with the remaining finger spreading part including the remaining finger support member that supports the remaining fingers.

The finger spread by the finger spreading part 420 may be fixed by pulling the finger fixing part 430 connected to the body 410 in a direction from an end of the finger toward the wrist and attaching the finger fixing part 430 to the body 410. The finger fixing part 430 may include a thumb finger fixing part and a remaining finger fixing part. For example, the thumb spread by the thumb spreading part may be fixed by pulling the thumb fixing part connected to the body 410 in a direction from an end of the thumb toward the wrist and attaching the thumb fixing part to the body 410. For example, the remaining fingers spread by the remaining finger spreading part may be fixed by pulling the remaining finger fixing part connected to the body 410 in a direction from an end of the fingers toward the wrist and attaching the remaining finger fixing part to the body 410. Accordingly, the rehabilitation glove according to the third embodiment of the inventive concept may spread and fix the finger of the user.

### Fourth Embodiment

Hereinafter, a rehabilitation glove according to a fourth embodiment of the inventive concept will be described.

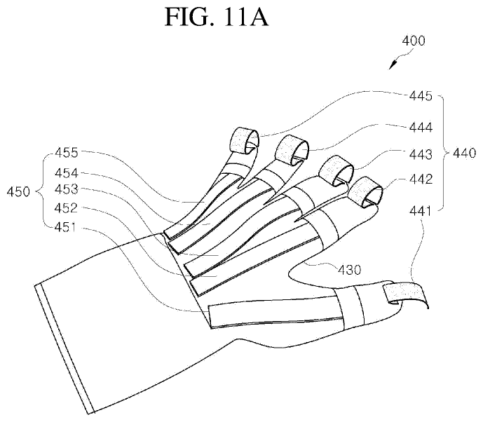

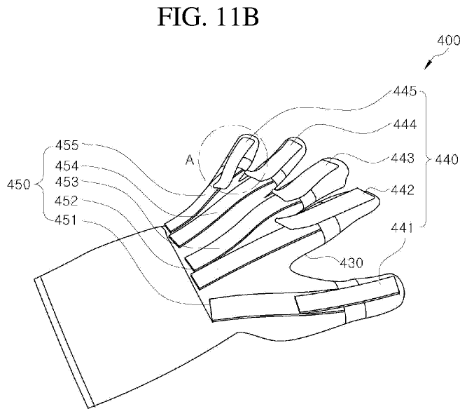

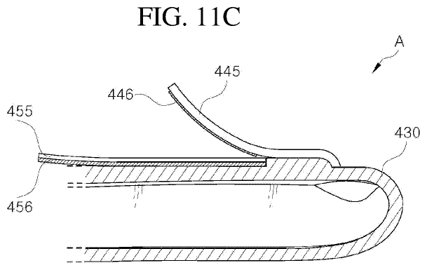

FIG. 11A is a schematic view of a hand insertion part included in the rehabilitation glove according to the fourth embodiment of the inventive concept, when the hand insertion part is viewed from a hand back while a finger spreading part is not fixed. FIG. 11B is a schematic view of the hand insertion part included in the rehabilitation glove according to the fourth embodiment of the inventive concept, when the hand insertion part is viewed from the hand back while the finger spreading part is fixed. FIG. 11C is a view schematically illustrating a cross-section of area A of FIG. 10B.

Referring to FIGS. 11A to 11C, the rehabilitation glove according to the fourth embodiment of the inventive concept has the configuration of the third embodiment, but the strap unit of the rehabilitation glove according to the fourth embodiment has a structure that is different from that of the third embodiment.

In the present embodiment, the strap unit is coupled to the body 430 and is formed to provide a tensile force in a direction, in which the joints of the finger of the user are spread at the hand back part of the user, and includes a finger spreading part 440 and a finger fixing part 450.

The finger spreading part 440 is formed to surround an end of the finger of the user. The finger spreading part 440 includes a Velcro fastener 446. The finger spreading part 440 may be attached to the finger fixing part 450 after the finger of the user is spread, and may fix the finger of the user. For example, the finger spreading part 440 may include a Velcro fastener, and may be attached to the finger fixing part 450 to spread the finger of the user.

The finger spreading part 440 may include the Velcro fastener 446, and may be detachably attached to the finger fixing part 450.

A thumb spreading part 441 may be attached to a thumb fixing part 451 after the thumb of the user is spread, and may fix the thumb of the user. An index finger spreading part 442 may be attached to an index finger fixing part 452 after the index finger of the user is spread, and may fix the index finger of the user. A middle finger spreading part 443 may be attached to a middle finger fixing part 453 after the middle finger of the user is spread, and may fix the middle finger of the user. A ring finger spreading part 444 may be attached to a ring finger fixing part 454 after the ring finger of the user is spread, and may fix the ring finger of the user. A little finger spreading part 445 may be attached to a little finger fixing part 455 after the little finger of the user is spread, and may fix the little finger of the user.

The finger fixing part 450 includes a Velcro fastener 456. The finger fixing part 450 is connected to the body 430. The finger fixing part 430 may include the Velcro fastener 456, and may be attached to the body 430 to fix the spread fingers with the finger spreading part 440.

The Velcro fastener 456 may include a first Velcro fastener, a second Velcro fastener, a third Velcro fastener, a fourth Velcro fastener, and a fifth Velcro fastener. The first Velcro fastener may be disposed in the thumb fixing part 451. The second Velcro fastener may be disposed in the index finger fixing part 452. The third Velcro fastener may be disposed in the middle finger fixing part 453. The fourth Velcro fastener may be disposed in the ring finger fixing part 454. The fifth Velcro fastener may be disposed in the little finger fixing part 455.

The finger fixing part 430 is detachably attached to the body 410. The finger fixing part 450 may be attached to the body 430 by applying a force in a direction from an end of the finger toward the wrist. The finger fixing part 450, for example, may be detachably attached to the hand back part of the body 430. The hand back part of the body 430 may include a Velcro fastener.

Referring to FIGS. 11A to 11C, the finger of the user may be surrounded by the finger spreading part 440 and may be spread by applying a force in a direction from the palm toward the hand back. The finger spread by the finger spreading part 440 may be fixed by pulling the finger fixing part 450 connected to the body 430 in a direction from an end of the finger toward the wrist and attaching the finger fixing part 450 to the body 430. Accordingly, the rehabilitation glove according to the fourth embodiment of the inventive concept may spread and fix the finger of the user.

The rehabilitation glove according to the fourth embodiment of the inventive concept may include a finger spreading part that spreads and fixes the finger of the user, and a finger fixing part that pulls and fixes the spread finger, and may help the user who requires a finger rehabilitation exercise to maximally spread the fingers and alleviate a spasticity phenomenon of the user.

Furthermore, the rehabilitation glove according to the fourth embodiment of the inventive concept may assist motion of the abnormal fingers of the users.

According to the inventive concept, a rehabilitation glove wearer may adjust strengths of the straps of the rehabilitation glove, which are formed in bone parts of fingers, such that all parts of the finger are spread, and thus may adjust the strengths that are necessary for the bone parts of the fingers as well as all the fingers.

Moreover, according to the inventive concept, when the straps formed in the bone parts of the fingers of the rehabilitation glove are pulled in a direction, in which the fingers of the user are extended, to be used, the support rings that support the extended straps are disposed to be inclined with respect to the fingers, and thus the tensile forces of the straps may be delivered more effectively.

Furthermore, according to the inventive concept, the straps may be formed in the fingers of the rehabilitation glove, and all portions of a hand, a palm, and a wrist of a wearer may be spread at once by the hand back strap, to which all the finger straps are attached.

The description of the inventive concept is exemplary, and it can be understood that those skilled in the art to which the inventive concept pertains can easily modify the inventive concept into other detailed forms without changing the technical spirits or the essential features.

Therefore, the above-described embodiments are exemplary in all aspects, and should be construed not to be restrictive. For example, the elements described in a single type may be distributed when being carried out, and the elements described as being distributed may be carried out while being coupled to each other.

The scope of the inventive concept is determined by the claims rather than the description of the inventive concept, and all changes or modifications derived from the meanings and scopes of the claims and the equivalents thereof are construed to be included in the scope of the inventive concept.

What is claimed is:

1. A rehabilitation glove comprising:
a body surrounding at least a portion of a hand of a user;
a finger spreading part connected to the body and configured to spread and fix a finger of the user; and
a finger fixing part configured to pull and fix the finger that is spread by the finger spreading part,
wherein the finger spreading part includes:
a support member configured to spread and fix a bone part of the finger of the user.

2. The rehabilitation glove of claim 1, wherein the support member supports the finger while contacting a back of the finger or a bottom of the finger of the user.

3. The rehabilitation glove of claim 2, wherein the finger spreading part spreads a knuckle of the finger by applying a force in a direction from a palm toward a hand back of the user.

4. The rehabilitation glove of claim 3, wherein the finger fixing part is connected to the body, and
wherein the finger fixing part includes a Velcro fastener, and is attached to the body to fix the spread finger.

5. The rehabilitation glove of claim 1, wherein the finger fixing part has elasticity.

6. The rehabilitation glove of claim 1, wherein the finger spreading part includes a Velcro fastener, and is attached to the finger fixing part to spread the finger of the user.

7. The rehabilitation glove of claim 6, wherein the finger fixing part is connected to the body, and
wherein the finger fixing part includes a Velcro fastener, and is attached to the body to fix the spread finger.

8. The rehabilitation glove of claim 1, wherein the finger fixing part is disposed to extend in a direction from an end of the finger toward a hand back of the user.

9. The rehabilitation glove of claim 1, further comprising:
a glove part surrounding the body,
wherein the glove part includes:
a hand back part surrounding a hand back of the user and a back of the finger; and
a palm part surrounding a bottom of the finger of the user and exposing at least a portion of a palm.

10. The rehabilitation glove of claim 1, wherein a hand back part of the body includes a Velcro fastener, and is detachably attached to the finger fixing part.

11. The rehabilitation glove of claim 9, wherein the glove part further includes:
a wrist fixing part surrounding a wrist.

12. The rehabilitation glove of claim 11, wherein the wrist fixing part includes a Velcro fastener.
